(19) United States
(12) Patent Application Publication
(10) Pub. No.: US 2026/0225444 A1
(43) Pub. Date: Aug. 6, 2026

(54) Method and System for Displaying a Video Call in a Vehicle

(71) Applicant: Bayerische Motoren Werke Aktiengesellschaft, Muenchen (DE)

(72) Inventors: Martin Wagner, Haimhausen (DE); Henning Hildebrandt, Muenchen (DE)

(21) Appl. No.: 19/151,577

(22) PCT Filed: Dec. 5, 2023
(86) PCT No.: PCT/EP2023/084280

§ 371 Date: Jul. 28, 2025

(30) Foreign Application Priority Data
Feb. 13, 2023 (DE) 10 2023 103 346.8

(51) Int. Cl. B60K 35/234; A61B 5/00; A61B 5/16; A61B 5/18; B60K 35/65; B60K 35/81; G06V 20/56; G06V 20/59; G06V 40/16; G07C 5/02; H04L 65/1089

(52) U.S. Cl. CPC B60K 35/234; A61B 5/0077; A61B 5/165; A61B 5/18; B60K 35/654; B60K 35/81; G06V 20/56; G06V 20/597; H04L 65/1089; B60K 2360/165; G06V 40/174; G07C 5/02

(57) ABSTRACT

A method for displaying a video telephone call in a vehicle includes receiving at the vehicle at least first image data corresponding to at least one first image having a depiction of a conversation participant remote from the vehicle. The method also includes ascertaining a travel situation of the vehicle and generating travel situation data corresponding to the travel situation. Second image data is generated that corresponds to a second image having a depiction of the conversation participant remote from the vehicle, based at least in part on the first image data received by the vehicle. The second image is displayed to a vehicle driver. The travel situation data is used for processing the first image data to generate the second image data, and/or setting at least one parameter of the displaying of the second image.

The present application is the U.S. national phase of PCT Application PCT/EP2023/084280 filed on Dec. 5, 2023, which claims priority of German patent application No. 10 2023 103 346.8 filed on Feb. 13, 2023, the entire contents of which are incorporated herein by reference.

### TECHNICAL FIELD

The disclosure relates generally to the field of vehicles, and more specifically, to the display of video telephone calls in a vehicle.

### BACKGROUND

DE 10 2018 208 513 A1 discloses a camera-monitor system for a motor vehicle having a telephony unit for a video telephony application. The camera-monitor system is designed to display a camera image of a conversation participant on monitor of the vehicle during the journey of the vehicle.

A telephone accessory having a generator for visual effects is known from US 2006/0183514 A1. The generator is designed to remove the background of an image, video, or other visual information and replace it with a new background.

US 2018/189550 A1 discloses a system for carrying out videoconferences. The system is designed to remove the background in an image of a conversation participant.

Driving a vehicle requires concentration on the surroundings of the vehicle by a vehicle driver, in particular on the traffic situation surrounding the vehicle. So as not to endanger the traffic safety, it is therefore important that a video telephone call is displayed to the vehicle driver such that the vehicle driver is distracted as little as possible from their driving task.

There is a need, therefore, for a method and a system for displaying a video telephone call in a vehicle, using which a video telephone call can be displayed in a traffic-safe manner during the journey without distracting a vehicle driver from their driving task.

### SUMMARY

The above-described need, as well as others, are met by one or more embodiment disclosed herein.

A first aspect is a method for displaying a video telephone call in a vehicle, at least one first image having a depiction of a conversation participant remote from the vehicle is acquired. First image data corresponding at least to the first image are generated. The first image data are transmitted to the vehicle. A travel situation of the vehicle is ascertained. Travel situation data corresponding to the travel situation are generated. Second image data, which correspond to a second image having a depiction of the conversation participant remote from the vehicle, are generated on the basis of the first image data transmitted to the vehicle. The second image is displayed to a vehicle driver of the vehicle by a display element of the vehicle. The first image data are processed here to generate the second image data taking into consideration the travel situation data. Alternatively or additionally, at least one parameter of the display of the second image is set on the basis of the travel situation data.

In the method, a video telephone call takes place between the vehicle driver and the conversation participant remote from the vehicle. During the video telephone call, the second image of the remote conversation participant is displayed to the vehicle driver. Especially visual information which is displayed to the vehicle driver during the journey can distract the vehicle driver from their driving task. The display of the second image therefore has an influence on the traffic safety. It has been recognized according to the invention that in previous methods for displaying a video telephone call in a vehicle, the current travel situation of the vehicle is not taken into consideration. Therefore, the effect of the video telephone call on the driving safety is also not taken into consideration. A goal conflict results in this way between the need of the vehicle driver to conduct the video telephone call, on the one hand, and the traffic safety, on the other hand. The present invention resolves this goal conflict in that the current travel situation of the vehicle is taken into consideration in the display of the second image. The current travel situation is in particular taken into consideration in that the second image is generated taking into consideration the travel situation. Known image processing methods can be applied to generate the second image. Specific examples of the generation of the second image are described hereinafter in conjunction with embodiments of the invention. Alternatively or additionally, modalities of the display of the second image are selected in accordance with the current travel situation. For this purpose, the at least one parameter of the display of the second image is set on the basis of the travel situation data. Specific examples of the at least one parameter are also described hereinafter in conjunction with embodiments of the invention.

In at least one embodiment, the travel situation data comprise at least one item of vehicle information related to the vehicle, in particular one of the following items of information: the speed of the vehicle, the degree of driver assistance of the vehicle, and the degree of autonomous driving of the vehicle. The higher the degree of driver assistance or autonomous driving is, the more visual information can be displayed to the vehicle driver without this driver being distracted from their task of driving the vehicle. This applies similarly to the speed of the vehicle. In particular when the vehicle is stationary, for example is parked, more visual information can be displayed to the vehicle driver. It can therefore be decided reliably using the vehicle information how the display of the second image is to take place without endangering the traffic safety.

In some embodiments, the travel situation data comprise at least one item of surroundings information related to the surroundings of the vehicle, in particular one of the following items of information: a traffic situation, a distance to a preceding vehicle, the current weather conditions, the current light conditions, and the current road conditions. The traffic situation can comprise, for example, whether the vehicle is located in congestion or how many further vehicles are located around the vehicle. The situation in the surroundings of the vehicle substantially determines the requirement for the attentiveness of the vehicle driver. Difficult weather conditions, poor light conditions or poor road conditions require a high level of concentration of the vehicle driver. The more attentiveness the surroundings situation requires of the vehicle driver, the less visual information can be displayed to the vehicle driver without endangering the traffic safety. It can therefore also be decided very reliably using the surroundings information how the display of the second image is to take place.

In some implementations, a characteristic value for the complexity of the travel situation is ascertained on the basis of the travel situation, wherein the travel situation data comprise the characteristic value for the complexity of the travel situation. The characteristic value for the complexity of the travel situation is ascertained in particular in consideration of the vehicle information and/or the surroundings information. The characteristic value can be ascertained in particular as a numeric value. Alternatively, the characteristic value can also be ascertained as a category or a degree of the complexity. The characteristic value specifies how complex the travel situation is, i.e. how high the requirement of the travel situation is for the concentration of the vehicle driver. Travel situations having high complexity, for example a high speed of the vehicle and bad road conditions, require a high level of concentration of the vehicle driver. The vehicle driver therefore cannot be additionally distracted by the display of the second image. Travel situations having lower complexity, for example when the vehicle drives autonomously and the road conditions are good, in contrast, require a level of concentration of the vehicle driver which is not as high. Without endangering the traffic safety, the display of the second image can demand more of the attention of the vehicle driver. On the basis of the characteristic value for the complexity of the travel situation, it can therefore be reliably decided how the display of the second image is to take place.

In at least one embodiment, at least one item of occupant information related to the vehicle driver is acquired. Occupant data corresponding to the occupant information are generated. The first image data are processed to generate the second image data taking into consideration the occupant data. Alternatively or additionally, the at least one parameter of the display of the second image is set taking into consideration the occupant data. In this embodiment, in addition to the current travel situation, the occupant information is also taken into consideration in the display of the second image. It can be directly determined on the basis of the occupant information, for example, whether the current display of the second image distracts the vehicle driver. A display of the second image can therefore be reliably selected on the basis of the occupant information, which is less distracting, thereby increasing traffic safety. Specific examples of the occupant information are described hereinafter in conjunction with exemplary embodiments of the invention.

In some embodiments, at least one driver image having a depiction of at least the face of the vehicle driver is acquired as the occupant information. Driver image data corresponding to the driver image are generated. It is ascertained on the basis of the driver image data whether the vehicle driver is distracted by the display of the second image. Furthermore, the occupant data comprise the information as to whether the vehicle driver is distracted by the display of the second image. For example, a pupil movement of the vehicle driver can be ascertained on the basis of the driver image data. States of concentration are expressed in a reduced pupil movement. It can therefore be ascertained from the pupil movement of the vehicle driver whether the vehicle driver is concentrating. Furthermore, a viewing direction of the vehicle driver can also be ascertained in particular from the driver image data. If the level of concentration of the vehicle driver is high while the vehicle driver looks at the second image, it can be presumed that the vehicle driver is distracted by the display of the second image. It can therefore be reliably determined on the basis of the driver image data whether the vehicle driver is distracted by the display of the second image.

In some implementations, at least one driver image having a depiction of at least the face of the vehicle driver is acquired as the occupant information and driver image data corresponding to the driver image are generated. A state of mind of the vehicle driver is ascertained on the basis of the driver image data. Furthermore, the occupant data comprise the ascertained state of mind. The state of mind can in particular be extracted from the driver image data using machine learning methods. Machine learning methods can reliably extract negative emotional expressions such as fatigue, stress, or concentration from image data. If such a negative emotional expression is recognized in the vehicle driver, the display of the second image can be adapted accordingly, for example a less distracting display can be selected if the vehicle driver is fatigued, stressed or is concentrating.

In some embodiments, at least one physiological variable of the vehicle driver is acquired as the occupant information. Physiological data corresponding to the physiological variable are generated, wherein the occupant data comprise the physiological data. Emotional states are accompanied by physiological reactions, for example an increased pulse rate or an increased breathing rate. These physiological reactions can be extracted from the physiological data. Emotional expression such as fatigue, stress or concentration can therefore be determined even more reliably on the basis of the physiological data.

In one or more embodiments, the second image is displayed semitransparent by a head-up display of the vehicle. The at least one parameter of the display of the second image is the degree of the transparency with which the second image is displayed. Up to a certain degree of the transparency, it is true that the display of the second image distracts all the less the higher the degree of the transparency is. It is therefore possible to react to particularly attention-intensive travel situations by setting the degree of the transparency. Vice versa, the degree of the transparency can also be reduced if the travel situation, in particular the light conditions, only permit the semitransparent displayed image to be recognized with difficulty. The distraction by the display of the second image is also reduced in this way.

In one or more implementations, the at least one parameter of the display of the second image is a display location within the vehicle interior, at which the second image is displayed. In particular, which display element of the vehicle the at least one image is displayed on can be set as the at least one parameter if the vehicle has multiple display units facing toward the vehicle driver. Alternatively or additionally, the at least one parameter can also be the size in which the second image is displayed. By setting the display location and/or the size of the second image in the field of view of the vehicle driver, how much of the attention of the vehicle driver the second image draws to itself can be influenced. A large display in the center of the field of view of the vehicle driver requires more attention than a small display in the periphery of the field of view of the vehicle driver. The distraction of the vehicle driver by the display of the second image can therefore also be reduced by setting the display location and/or the size of the second image in the field of view of the vehicle driver.

In some embodiments, an additional item of image information of the first image is ascertained on the basis of the first image data. Image information data corresponding to the additional image information are generated. The first image data are processed to generate the second image data in consideration of the image information data. Alternatively or additionally, the at least one parameter of the display of the second image is set in consideration of the image information data. In this embodiment, in addition to the current travel situation, the additional image information is also taken into consideration in the display of the second image. For example, it can be determined on the basis of the additional image information whether the second image, which is generated from the first image, can be displayed in a distraction-free manner, or whether the modalities of the display of the second image have to be adapted to enable a distraction-free display. Specific examples of the additional image information are mentioned hereinafter on the basis of further exemplary embodiments.

In one or more embodiments, a characteristic value for the emotionality of the first image is ascertained on the basis of the first image data as the additional image information. The image information data comprise the characteristic value for the emotionality of the first image. The characteristic value for the emotionality of the first image can be ascertained in particular with the aid of machine learning methods. The characteristic value can be a numeric value. Alternatively, the characteristic value is one or more emotions which can be ascertained from the first image with the aid of the machine learning method. Particularly emotional images or images having particularly strong emotions, for example anger, stress and excitement, can distract the vehicle driver. If the emotional content of the image is taken into consideration in the display of the second image, the display of the second image can be adapted so that the vehicle driver is not additionally distracted by the emotional content of the image. For example, the second image can be generated, in particular using a machine learning method, so that the emotional content of the second image is weakened. The display of the second image can therefore take place in an even more traffic-safe manner.

In one or more implementations, a first image sequence of multiple chronologically successive first images, each of which comprises a depiction of the conversation participant remote from the vehicle, is acquired. The first image data are generated corresponding to the first image sequence. The second image data correspond to a second image sequence of multiple chronologically successive second images each having one of the depictions of the remote conversation participant. A characteristic value for the dynamics of the first image sequence can be ascertained as the additional image information on the basis of the first image data, wherein the image information data comprise the characteristic value for the dynamics of the first image sequence. Particularly rapid movements, in particular in the periphery of the field of view, trigger instinctive reactions which can be very distracting. Such movements therefore have to be filtered out of the second image sequence before it can be displayed to the vehicle driver if this driver cannot be additionally distracted by the display of the second image sequence due to the travel situation. For example, the dynamics of the second image sequence are reduced in relation to the first image sequence on the basis of the ascertained dynamics if the ascertained characteristic value of the dynamics of the first image sequence is above a predetermined threshold value. The predetermined threshold value can be determined here on the basis of the current travel situation. The reduction of the dynamics can take place, for example, with the aid of a suitable machine learning method.

In some implementations, a background is ascertained in the first image on the basis of the first image data. The second image data are generated on the basis of the ascertained background such that the background is displayed transparent in the second image. In this embodiment, the background of the depiction of the remote conversation participant is removed in the second image, i.e. the remote conversation participant is cropped in the second image. This reduces the display of the remote conversation participant to the essential features, in particular if the second image is displayed by a head-up display. The second image is therefore displayed to the vehicle driver so that the vehicle driver is distracted as little as possible from the traffic situation by the display of the second image.

Another aspect of the disclosure relates to a system for displaying a video telephone call. The system comprises an image acquisition unit, which is designed to acquire at least one first image having a depiction of a conversation participant remote from the vehicle and to generate first image data at least corresponding to the image. A travel situation acquisition unit of the system is designed to ascertain a travel situation of the vehicle and to generate travel situation data corresponding travel situation. The system moreover comprises a display element of the vehicle, which is arranged in the vehicle interior and is designed to display visual information to a vehicle driver of the vehicle. The system furthermore comprises a control unit of the vehicle, which is designed to receive the first image data, to generate second image data, which correspond to a second image having the depiction of the conversation participant remote from the vehicle, on the basis of the first image data, and to control the display element such that the display element displays the second image to the vehicle driver of the vehicle. The control unit can be designed to process the first image data to generate the second image data taking into consideration the travel situation data. Alternatively or additionally, the control unit can be designed to set at least one parameter of the display of the second image on the basis of the travel situation data.

The system has the same advantages as the embodiments of the method discussed above. In particular, the system can be refined using the features of the dependent claims directed to the method. Furthermore, the above-described method can be refined using the features described in conjunction with the system.

The travel situation acquisition unit can be designed to acquire at least one movement parameter of the vehicle. The movement parameter can be, for example, the speed of the vehicle or a distance of the vehicle to a preceding vehicle. However, the movement parameter can also be a derived variable which characterizes, for example, how often the vehicle changes lanes or how well the vehicle acquisition unit can also comprise a navigation unit of the vehicle, which is designed to provide navigation data of the vehicle. The navigation unit can be a navigation unit of the vehicle. Alternatively, the navigation unit can also be a part of the mobile terminal introduced into the vehicle by the vehicle driver. The navigation data are movement data of the vehicle and in particular comprise items of information about a route covered and/or a speed of the vehicle, i.e. information about movement parameters of the vehicle. The travel situation acquisition unit can ascertain from the navigation data, for example, whether the vehicle is currently traveling on a monotonous route.

The travel situation acquisition unit can furthermore comprise an image acquisition unit of the vehicle, in particular a camera of the vehicle, which is designed to acquire at least one image having a depiction of at least one area outside the vehicle, in particular an area in front of the vehicle. The image acquisition unit is designed in particular to acquire a two-dimensional image of the area outside the vehicle. The image acquisition unit in particular acquires light in the optical spectrum and/or in the infrared spectrum. Alternatively to an optical camera, the image acquisition unit can also comprise a RADAR or LIDAR system, which generates a three-dimensional image of the area outside the vehicle, in particular in the form of a point cloud. The travel situation acquisition unit can in particular extract information about the current traffic situation from image data acquired by the image acquisition unit. For example, the travel situation acquisition unit can extract from the image data whether the vehicle is located in congestion or how many further vehicles are located around the vehicle. The travel situation can be ascertained very reliably from this information.

The display element of the vehicle is in particular a display screen facing toward the vehicle driver, a head-up display, a central information display or a graphic instrument cluster of the vehicle.

The above-described features and embodiments, as well as others will become more readily apparent to those of ordinary skill in the art by reference to the following detailed description and accompanying drawings.

### BRIEF DESCRIPTION OF THE DRAWINGS

FIG. 1 shows a schematic representation of a system for displaying a video telephone call in a vehicle according to an exemplary embodiment; and

FIG. 2 shows a flow chart of a method for displaying the video telephone call in the vehicle.

### DETAILED DESCRIPTION

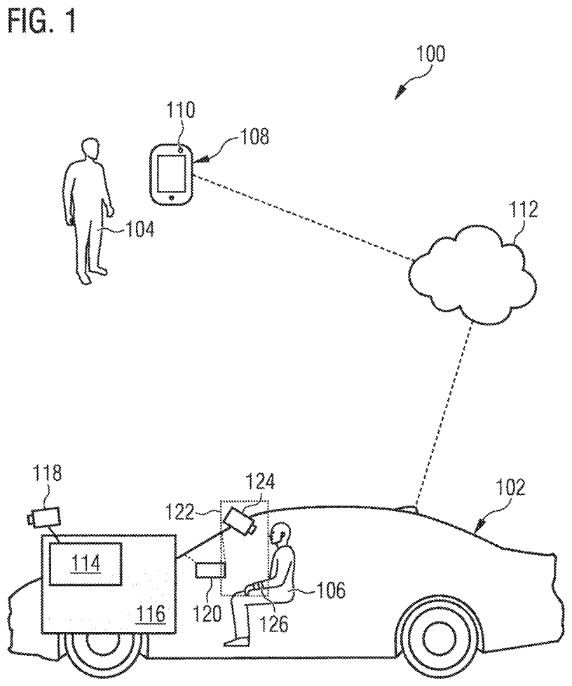

FIG. 1 shows a schematic representation of a system 100 for displaying a video telephone call in a vehicle 102 according to an exemplary embodiment.

The system 100 enables a video telephone call between a conversation participant 104 remote from the vehicle 102 and a vehicle driver 106 of the vehicle 102. In the exemplary embodiment shown, the remote conversation participant 104 participates, solely by way of example, in the video telephone call with the aid of a smart device 108. Alternatively, the remote conversation participant 104 can also, for example, participate in the video telephone call from a further vehicle or from a fixed PC workstation.

The system 100 comprises an image acquisition unit 110, which is embodied in the exemplary embodiment shown as a camera of the smart device. The image acquisition unit 110 is designed to acquire at least one first image of the remote conversation participant 104 and to generate image data corresponding to the first image. The first image comprises a depiction of the remote conversation participant 104, in particular of the face of the remote conversation participant 104. In particular, the image acquisition unit 110 is designed to acquire a first image sequence of chronologically successive first images, i.e. a video sequence, an image stream or a video stream of the remote conversation participant 104. In the exemplary embodiment shown in FIG. 1, the first image data, possibly together with further audio, image and/or video data of the video call, are transmitted to the vehicle 102. The first image data are in particular transmitted via a data remote transmission network 112, for example the Internet. However, other computer networks such as LAN, MAN or WAN networks or mobile wireless networks can also be used for the transmission of the first image data.

The system 100 moreover comprises a travel situation acquisition unit 114 of the vehicle 102, which is shown solely by way of example in FIG. 1 as part of a control unit 116 of the vehicle 102. The travel situation acquisition unit 114 comprises, solely by way of example, an exterior camera 118 of the vehicle 102, which is arranged and aligned so as to acquire an image having a depiction of at least an area in front of the vehicle 102. The travel situation acquisition unit 114 is moreover designed to receive items of vehicle information related to the vehicle 102 from the control unit 116 of the vehicle 102, for example the currently set degree of driver assistance or autonomous driving. The travel situation acquisition unit 114 can comprise further elements of the vehicle 102, for example a navigation unit, which provides navigation data related to the vehicle 102, and/or further sensors of the vehicle 102, which can provide items of vehicle information related to the vehicle 102 or items of surroundings information related to the surroundings of the vehicle 102. One example of a sensor which can provide an item of vehicle information related to the vehicle 102 is a speedometer of the vehicle. One example of a sensor which can provide an item of surroundings information related to the surroundings of the vehicle 102 is a distance sensor of the vehicle 102. The travel situation acquisition unit 114 generates travel situation data from the acquired travel situation, i.e. the set of all items of vehicle information and/or surroundings information acquired and received by the travel situation acquisition unit 114, and transmits these travel situation data to the control unit 116.

The control unit 116 of the vehicle 102 is part of the system 100 and is designed to receive the first image data and the travel situation data and to generate second image data, which correspond to a second image having the depiction of the conversation participant 104 remote from the vehicle 102, on the basis of the first image data. When the first image sequence is acquired, the control unit 116 generates the second image data in accordance with a second image sequence of second images, each of which comprises a depiction of the remote conversation participant 104. The control unit 116 is furthermore designed to control a display element 120 of the vehicle 102 such that the display element 120 displays the second image or the second image sequence to the vehicle driver 106 of the vehicle 102. In particular, the control unit 116 is also designed to process the first image data to generate the second image data taking into consideration the travel situation data, and/or to set at least one parameter of the display of the second image on the basis of the travel situation data.

The control unit 116 can take into consideration further data for generating the second image data and/or for setting the at least one parameter of the display of the second image. For example, the control unit 116 can take into consideration occupant data, which correspond to an item of occupant information related to the vehicle driver 106 and are acquired by an occupant acquisition unit 122. Furthermore, the control unit 116 can take into consideration image information data, which correspond to an item of additional image information obtained from the first image. For example, the control unit 116 can be designed to ascertain a characteristic value for the dynamics of the first image sequence and to generate the second image data such that the dynamics of the second image sequence are reduced. The control unit 116 can also ascertain a characteristic value for the emotionality of the first image sequence, for example in the form of a list of emotions which are contained in the first image or the first image sequence. The control unit 116 can then generate the second image data such that particularly negative and therefore distracting items of information are filtered out of the second image or the second image sequence.

In the embodiment shown in FIG. 1, the system 100 comprises the occupant acquisition unit 122, which is designed to acquire an item of occupant information related to the vehicle driver 106. Solely by way of example, the occupant acquisition unit 122 comprises an interior camera 124 of the vehicle 102 directed toward the vehicle driver 106. The interior camera 124 acquires a driver image having a depiction of the vehicle driver 106 as one of the items of occupant information. The control unit 116 can ascertain, for example, from driver image data, which are generated from the driver image, whether the vehicle driver 106 is distracted by the display of the second image or the second image sequence, and if necessary can adapt the display of the second image or the second image sequence.

The occupant acquisition unit 122 furthermore comprises a smart device 126 introduced by the vehicle driver 106 into the vehicle interior, which is shown solely by way of example as a smart watch. The smart device 126 acquires an item of physiological information, for example the pulse rate, of the vehicle driver 106 as one of the items of occupant information. Physiological data, which the control unit 116 can also consider for the adaptation of the display of the second image or the second image sequence, are generated from the physiological information.

The display element 120 of the vehicle 102 is arranged in the vehicle interior and is designed to display visual information to the vehicle driver 106 of the vehicle 102. Solely by way of example, the display element 120 is shown as a head-up display of the vehicle 102. Alternatively, the display element 120 can also be a display screen facing toward the vehicle driver 106, a central information display or a graphic instrument cluster of the vehicle 102. The control unit 116 can set a display location within the vehicle interior as the at least one parameter. This means in particular that the control unit 116 is designed to set which of the display elements 120 of the vehicle 102 the second image or the second image sequence is displayed on at least on the basis of the travel situation data.

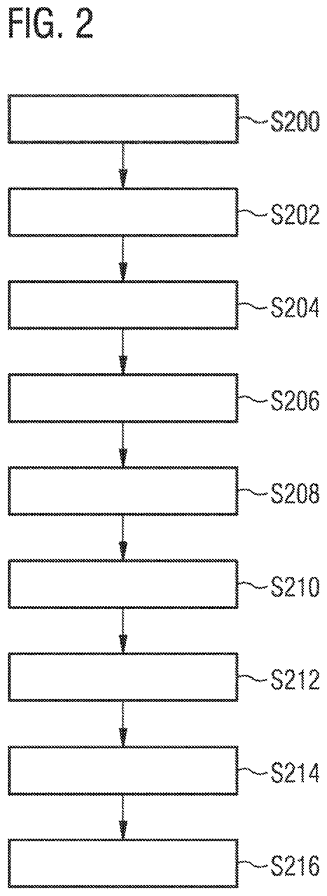

FIG. 2 shows a flow chart of a method for displaying a video telephone call in the vehicle 102.

In step S200, the method is started. In step S202, the image acquisition unit 110 acquires at least the first image having the depiction of the conversation participant 104 remote from the vehicle 102. In particular, in this step the image acquisition unit 110 acquires the multiple chronologically successive first images, which are part of the first image sequence. The image acquisition unit 110 furthermore generates in this step the first image data corresponding to the first image or the first image sequence. In step S204, the first image data are transmitted to the vehicle 102.

In step S206, the control unit 116 actuates the travel situation acquisition unit 114 to acquire the travel situation. The travel situation acquisition unit 114 acquires and/or receives in this case in particular the items of vehicle information and the items of surroundings information and processes them in order to ascertain the travel situation. The travel situation acquisition unit 114 then generates the travel situation data from the ascertained travel situation and transmits these data to the control unit 116. In optional step S208, the control unit 116 ascertains the additional image information of the first image on the basis of the first image data and generates the image information data corresponding to the additional image information. In optional step S210, the control unit 116 actuates the occupant acquisition unit 122 to acquire the occupant information. The occupant acquisition unit 122 generates the occupant data from the occupant information and transmits these data to the control unit 116. Steps S206 to S210 can be carried out simultaneously or following one another in any sequence. Second ones of steps S206 to S210 can also be carried out simultaneously while a third one of steps S206 to S210 is carried out before or after.

In step S212, the control unit 116 processes the first image data to generate the second image data at least taking into consideration the travel situation data. In addition, the control unit 116 can also take into consideration the image information data and/or the occupant data, if these were generated in step S208 or step S210, respectively, to generate the second image data in step S212. In step S214, the control unit 116 sets the at least one parameter of the display of the second image on the basis of the travel situation data. In addition, the control unit 116 can also take into consideration the image information data and/or the occupant data in step S214 in order to set the at least one parameter if these data were generated in step S208 or step S210, respectively. In the method according to FIG. 2, at least one of the two steps S212 and S214 is carried out.

In step S216, the control unit 116 actuates one of the display elements 120 of the vehicle 102 in order to display the second image to the vehicle driver 106 of the vehicle 102. In step S218, the method is ended.

In the exemplary embodiment described on the basis of FIGS. 1 and 2, at least the image acquisition unit 110, the travel situation acquisition unit 114, the display element 120 of the vehicle 102, and the control unit 116 of the vehicle 102 form the system 100 for displaying the video telephone call in the vehicle 102. Further elements and features shown in FIGS. 1 and 2 and mentioned in the preceding description can be part of the system 100 for displaying the video telephone call in the vehicle 102.

### LIST OF REFERENCE NUMERALS

100 system
102 vehicle
104 conversation participant
106 vehicle driver
108 smart device
110 image acquisition unit
112 data remote transmission network
114 travel situation acquisition unit
116 control unit
118 camera
120 display element
122 occupant acquisition unit
124 camera
126 smart device

1.-15. (canceled)

16. A method for displaying a video telephone call in a vehicle, the method comprising:
receiving at the vehicle at least first image data corresponding to at least one first image having a depiction of a conversation participant remote from the vehicle;
ascertaining a travel situation of the vehicle and generating travel situation data corresponding to the travel situation;
generating second image data, which correspond to a second image having a depiction of the conversation participant remote from the vehicle, based at least in part on the first image data received by the vehicle;
displaying the second image to a vehicle driver of the vehicle using a display element of the vehicle;
wherein the travel situation data is used for at least one of the group consisting of:
processing the first image data to generate the second image data based at least in part on the travel situation data; and
setting at least one parameter of the displaying of the second image based at least in part on the travel situation data.

17. The method as claimed in claim 16, wherein the travel situation data comprises at least one item of vehicle information regarding the vehicle.

18. The method as claimed in claim 17, wherein vehicle information includes a speed of the vehicle, a degree of driver assistance of the vehicle, and a degree of autonomous driving of the vehicle.

19. The method as claimed in claim 16, wherein the travel situation data comprise at least one item of surroundings information related to surroundings of the vehicle,

20. The method as claimed in claim 19, wherein the surroundings information includes a traffic situation, a distance to a preceding vehicle, current weather conditions, current light conditions, and current road conditions.

21. The method as claimed in claim 16, wherein a characteristic value for a complexity of the travel situation is ascertained based at least in part on the travel situation, wherein the travel situation data comprise the characteristic value for the complexity of the travel situation.

22. The method as claimed in claim 16, further comprising acquiring at least one item of occupant information related to the vehicle driver and generating occupant data corresponding to the occupant information; and
wherein the first image data are processed to generate the second image data taking into consideration the occupant data; and/or
wherein the at least one parameter of the display of the second image is set taking into consideration the occupant data.

23. The method as claimed in claim 22, wherein at least one driver image having a depiction of at least a face of the vehicle driver is acquired as the occupant information and driver image data corresponding to the driver image are generated, and the method further comprises:
ascertaining based at least in part on the driver image data whether the vehicle driver is distracted by the display of the second image;
wherein the occupant data comprise the information as to whether the vehicle driver is distracted by the display of the second image.

24. The method as claimed in claim 22, wherein at least one driver image having a depiction of at least a face of the vehicle driver is acquired as the occupant information and driver image data corresponding to the driver image are generated, and the method further comprises:
determining a state of mind of the vehicle driver based at least in part on the driver image data;
wherein the occupant data comprise the determined state of mind.

25. The method as claimed in claim 22, wherein at least one physiological variable of the vehicle driver is acquired as the occupant information and physiological data corresponding to the physiological variable are generated, wherein the occupant data comprise the physiological data.

26. The method as claimed in claim 16, wherein the second image is displayed with a degree of transparency by a head-up display of the vehicle and the at least one parameter of the display of the second image is the degree of the transparency.

27. The method as claimed in claim 16, wherein:
the travel situation data is used for setting the at least one parameter of the displaying of the second image based at least in part on the travel situation data; and
the at least one parameter of the displaying of the second image is a display location within a vehicle interior at which the second image is displayed.

28. The method as claimed in claim 16, wherein:
the travel situation data is used for setting the at least one parameter of the displaying of the second image based at least in part on the travel situation data; and
the at least one parameter of the displaying of the second image is a size in which the second image is displayed.

29. The method as claimed in claim 16, wherein:
an additional item of image information of the first image is ascertained based at least in part on the first image data and image information data corresponding to the additional image information are generated;
wherein the image information data is used for at least one of the group consisting of:
processing the first image data to generate the second image data based at least in part on the image information data; and
setting at least one parameter of the displaying of the second image based at least in part on the image information data.

30. The method as claimed in claim 29, wherein the addition image information comprises an emotionality of the first image; and
wherein the image information data comprise a characteristic value for the emotionality of the first image.

31. The method as claimed in claim 16, wherein a first image sequence of multiple chronologically successive first images, each of the successive first images comprises a depiction of the conversation participant remote from the vehicle, is received by the vehicle, and wherein the first image data are generated in accordance with the first image sequence;
wherein the second image data correspond to a second image sequence of multiple chronologically successive second images, each of the successive second images having one of the depictions of the conversation participant remove from the vehicle; and
wherein a characteristic value for dynamics of the first image sequence is ascertained as additional image information based at least in part on the first image data, wherein the image information data comprise the characteristic value for the dynamics of the first image sequence.

32. The method as claimed in claim 16, wherein a background in the first image is ascertained based at least in part on the first image data; and in which the second image data are generated based at least in part on the ascertained background such that the background is displayed transparent in the second image.

33. A system for displaying a video telephone call in a vehicle, comprising:
an image acquisition unit configured to acquire at least one first image having a depiction of a conversation participant remote from the vehicle and to generate first image data corresponding at least to the image;
a travel situation acquisition unit configured to determine a travel situation of the vehicle and to generate travel situation data corresponding to the travel situation;
a display element of the vehicle in a vehicle interior and configured to display visual information to a vehicle driver of the vehicle; and
a control unit of the vehicle configured to:
receive the first image data, to generate second image data, which correspond to a second image having the depiction of the conversation participant remote from the vehicle, based at least in part on the first image data; and
control the display element such that the display element displays the second image to the vehicle driver of the vehicle;
use the travel situation data for at least one of the group consisting of:
processing the first image data to generate the second image data based at least in part on the travel situation data; and
setting at least one parameter of the displaying of the second image based at least in part on the travel situation data
